(19) United States
(12) Patent Application Publication
(10) Pub. No.: US 2006/0153925 A1
(43) Pub. Date: Jul. 13, 2006

(54) Novel solid pharmaceutical composition comprising amisulpride

(75) Inventors: Frederic Andre, Varrieres-Le-Buisson (FR); Henri Da Cruz, Le Mee-Sur-Seine (FR); Michel Denni, Morangis (FR); Gareth Lewis, Dourdan (FR); Jerome Mignonneau, Saint-Clement-Des-Levees (FR); Agnes Ribardiere, Fontenay-Aux-Roses (FR)

Correspondence Address: ROSS J. OEHLER;AVENTIS PHARMACEUTICALS INC., 1041 ROUTE 202-206, MAIL CODE: D303A, BRIDGEWATER, NJ, 08807, US

(73) Assignee: Sanofi-Aventis, Paris (FR)

(21) Appl. No.: 11/327,603

(22) Filed: Jan. 6, 2006

Related U.S. Application Data
(63) Continuation of application No. PCT/FR04/01792, filed on Jul. 8, 2004.

(30) Foreign Application Priority Data
Jul. 9, 2003 (FR) 03/08409

(51) Int. Cl. A61K 9/50; A61K 9/16

(52) U.S. Cl. 424/490

(57) ABSTRACT

The invention relates to a solid pharmaceutical composition for oral administration of amisulpride, which comprises at least one coated amisulpride particle and at least one pharmaceutically acceptable excipient suitable for an orodispersible administration of the composition.

### CROSS-REFERENCE TO RELATED APPLICATIONS

This application is a continuation of PCT/FR2004/001792, filed Jul. 8, 2004, which claims priority from French Patent Application No 03/08409, filed Jul. 09, 2003.

### SUMMARY OF THE INVENTION

The present invention relates to a novel solid pharmaceutical composition comprising amisulpride, this composition being in oral dispersible form.

### BACKGROUND OF THE INVENTION

Amisulpride, or 4-amino-N-[(1-ethyl-2-pyrrolidin-yl)methyl]-5-(ethylsulfonyl)-2-methoxybenzamide, its isomers and certain derivatives thereof are described in U.S. Pat. No. 4,401,822 of Thominet et al.

Amisulpride is an atypical antipsychotic agent used in the treatment of psychoses, more particularly in the treatment of paranoid and productive schizophrenias or acute delirious psychoses, and also in the treatment of schizophrenia deficiency states, residual psychotic changes and inhibitory states with slowing. Amisulpride is also useful in the treatment of dysthymia.

Amisulpride is known to be administered orally, generally in the form of tablets each containing a 100, 200 or 400 mg dose (Vidal, edition 2003, Solian® monograph on pages 1736 and 1738).

The daily doses of amisulpride administered orally are, however, often high and may be up to 1200 mg/day during acute psychotic episodes.

Patients treated with amisulpride must therefore ingest several tablets daily.

However, some of these patients, due to the very fact of their pathological state, may encounter difficulties, or even show pronounced reticence toward regularly ingesting a large number of tablets and thus in correctly adhering to their prescription.

It has thus been sought to develop novel oral forms of amisulpride.

It was first envisaged to develop tablets comprising a higher dose of amisulpride, for example doses of greater than 600 mg. However, such tablets were found to be too large to be easily swallowed by the patients. Studies showed that a range of tablets containing 100 mg, 200 mg and 400 mg doses of amisulpride remained the most appropriate.

It was then envisaged to prolong and/or intensify the gastrointestinal absorption of amisulpride in order especially to reduce the number of daily intakes of amisulpride tablets, while at the same time maintaining, or even improving, the therapeutic efficacy of this active principle. Mention may be made, in particular, of WO 00/51563, which describes a composition comprising a combination of a benzamide (especially amisulpride) and of an absorption promoter (especially a P-glycoprotein inhibitor), this combination being intended to improve the intestinal absorption of the benzamide, i.e. to increase the fraction of benzamide that crosses the intestinal barrier (page 1, lines 21-23), and thus to be able to reduce the administered doses for a given effective dose and to reduce the number of daily intakes (page 2, lines 23-26).

However, irrespective of the number of intakes, it was found that the main problem for the amisulpride active principle consisted in being able to ensure that the tablets were correctly taken by the patients.

This problem of adherence to the prescriptions is in fact entirely crucial, given the specific pathologies to be treated, especially in the case of the most reticent patients requiring the intervention of an external aid to ensure good quality of the intake, i.e. effective swallowing of the amisulpride tablet.

The reason for this is that it has been found that reticent patients have a tendency to simulate the taking of the amisulpride tablet, especially by swallowing the water given to them, while keeping the tablet hidden in their oral cavity, to get rid of it later by spitting it out.

The fact of the matter is that, most particularly in a hospital environment, only a limited amount of time can be devoted per patient for this external aid, which makes this type of simulation easier, thus constituting a real problem for the health of the patient.

Another problem associated with this active principle amisulpride lies in its very great bitterness.

In order to be able to quantify the magnitude of this bitterness, a panel of 6 individuals made it possible to find that the bitterness of amisulpride is detected with a tablet containing 1 mg of amisulpride that can be released in the mouth. This bitterness becomes unacceptable when the tablet contains 8 mg of amisulpride that can be released in the mouth.

The degree of interest of an administration of amisulpride at doses of from 100 to 400 mg orally, which would make it possible to avoid any simulation without, however, having a sensation of bitterness in the mouth that is unacceptable as regards any swallowing, is thus measured in order to obtain a real improvement in the adherence to the prescription, in particular in the case of the most reticent patients.

This problem of adherence to the prescription should, however, also be taken into account in the case of the least reticent patients that the therapists have managed to stabilize often after several months of a correctly followed treatment.

The reason for this is that it is accepted by the therapists of patients already treated with amisulpride, in particular schizophrenic patients, that a simple change in the appearance or the modes of taking of the medicament may result in a dangerous destabilization of these patients.

Finally, it is also accepted by therapists that any change in the pharmaceutical composition for an active principle such as amisulpride should preferably make it possible to conserve, besides the appearance of the medicament, the pharmacokinetics advantageously afforded by the tablets already used by the patients in order to prevent any destabilization. Now, it has been found that the active principle amisulpride, when it is to be administered orally, has limited bioavailability (of about 48%), which may be attributed to partial gastrointestinal absorption of this active principle. Thus, certain modifications to the release profile might result in insufficient passage to the brain.

### DETAILED DESCRIPTION OF THE INVENTION

A novel solid pharmaceutical composition for oral administration of amisulpride has now been found, entirely surprisingly and unexpectedly, which makes it possible to solve the specific problems of this active principle.

This novel composition in particular constitutes a genuine improvement in the treatment of all the patients concerned, insofar as it makes it possible to significantly improve the adherence to the prescription by the most reticent patients while at the same time remaining useable by the least reticent patients.

The present invention thus relates to a novel solid pharmaceutical composition for oral administration of the active principle amisulpride, characterized in that it comprises at least one coated amisulpride particle and at least one pharmaceutically acceptable excipient suitable for an orodispersible administration of the composition.

According to the present invention, the term “orodispersible administration” means an oral administration by rapid disintegration of the solid composition (especially a tablet) in the mouth, preferably in less than 30 seconds, without the need to chew or to supply water in order to swallow the disintegrated composition, the composition needing to be able to be swallowed preferably in less than 90 seconds after placing in the mouth. According to the present invention, the term “orodispersible administration” thus in particular means an oral administration by buccal disintegration of the solid composition before being swallowed by the patient, as opposed to a “standard” oral administration, i.e. an oral administration in which the patient swallows a non-disintegrated solid composition (especially a standard tablet) before the start of any disintegration, for example in the stomach. A person skilled in the art thus understands in particular that the envelope of a standard gel capsule or of a sachet cannot be considered as a pharmaceutically acceptable excipient suitable for such an orodispersible administration.

According to the present invention, the term “amisulpride” means (R,S)-amisulpride, i.e. the racemic mixture (for example the product manufactured by the company Finorga), present in the pharmaceutical form distributed by the company Sanofi-Synthelabo under the name Solian®, a levorotatory or dextrorotatory isomer of amisulpride, an amisulpride derivative or a derivative of one of its levorotatory or dextrorotatory isomers, this derivative being chosen from an addition salt with a pharmaceutically acceptable acid, a quaternary ammonium salt or an oxide of amisulpride or of one of its levorotatory and dextrorotatory isomers, or a mixture thereof.

According to the present invention, the term “amisulpride particle” means a particle comprising amisulpride, this particle possibly being in perfect or imperfect spherical or spheroidal form, or in any other particle form known to those skilled in the art, as obtained via a granulation process (wet granulation, granulation with a meltable binder, granulation by mounting with powders and/or liquids, by rotogranulation and granulation by extrusion-spheronization), a fluidization process, an atomization process, a prilling process or via an amisulpride crystallization process. A person skilled in the art thus understands that this amisulpride particle may comprise only amisulpride, especially when it is obtained via a crystallization process, or may also comprise at least one agent resulting from the implementation of the process for forming this amisulpride particle, especially a spheronization excipient when this amisulpride particle is obtained via a granulation process, as described below.

The solid composition according to the invention may, of course, comprise more than one coated amisulpride particle, in the form of a mixture with said at least one pharmaceutically acceptable excipient suitable for orodispersible administration of the composition.

The coated amisulpride particle preferably consists of an amisulpride particle, as defined above, having at its surface a coating chosen from the group consisting of lipid coatings and polymer coatings.

The lipid and polymer coating processes that may be used to coat amisulpride particles are well known to those skilled in the art.

Lipid coating, also known as “hot-melt coating”, is a process of hot coating of a particle by spraying a hot-melt coating material thereon, for example an oil or a wax. It may be advantageously performed in a fluidized air bed machine especially of GPCG1 type as sold by the company Glatt.

Polymer coating, which is performed in a known manner with aqueous or organic vehicles, is a process of coating a particle by spraying thereon an aqueous or organic solution or dispersion of a polymer and of known additives such as a plasticizer, especially steric acid, triethyl citrate and/or dibutyl sebacate, and/or a nonstick agent such as talc, a silica derivative, such as Syloid® (silica gel), or magnesium stearate. The coating step may be followed by a maturation step for evaporating off the residual solvents and for completing the formation of the coating film. This polymer coating process may be performed in a fluidized air bed machine.

When the amisulpride particle comprises a lipid coating, this lipid coating preferably comprises a lipid material chosen from the group consisting of:
mono-, di- and triglycerides of fatty acids formed from chains containing from 6 to 32 carbon atoms, which are preferably saturated, such as glyceryl monostearate, glyceryl distearate, hydrogenated cottonseed oil, hydrogenated palm oil, hydrogenated soybean oil, hydrogenated castor oil and, more generally, hydrogenated plant oils. Hydrogenated castor oil, known under the name glyceryl tris(12-hydroxystearate) (RN CAS 8001-78-3), is particularly preferred, such as the product sold under the name Cutina HR®;
polyglyceryl monoesters and diesters, such as diglyceryl monostearate, triglyceryl monostearate, hexaglyceryl dipalmitate and decaglyceryl distearate;
polyethylene glycol monoesters and diesters, in particular mixtures thereof with mono-, di- and triglycerides, such as the products sold under the respective names “Gelucire 50/13®” and “Gelucire 50/02®” by the company Gattefosse. A mixture of “Gelucire 50/13®∞ and of “Gelucire 50/02®” may more particularly be used;
propylene glycol esters, such as polyethylene glycol monostearate;
fatty acid esters of sucrose, formed from chains containing from 6 to 32 carbon atoms, such as sucrose monostearate or sucrose monopalmitate;
fatty acids and fatty alcohols, formed from chains containing from 6 to 32 carbon atoms, such as stearic acid, cetyl alcohol or stearyl alcohol;
plant waxes, animal waxes, petroleum waxes and synthetic waxes, such as jojoba wax, carnauba wax, cocoa butter, beeswax, lanolin or paraffin wax;
   and mixtures thereof.

When the amisulpride particle comprises a polymer coating, this coating preferably comprises a polymer chosen from the group consisting of cellulose-based polymers, acrylic polymers, vinyl polymers and carboxyvinyl polymers, and mixtures thereof.

When the polymer coating comprises a cellulose-based polymer, this polymer may advantageously be chosen from the group consisting of ethylcellulose, hydroxyethylcellulose, hydroxypropylcellulose, hydroxypropylmethylcellulose and carboxymethylcellulose, and mixtures thereof. Ethylcellulose in aqueous dispersion may be used in particular, such as the product sold under the name Aquacoat® by the company FMC BioPolymer.

Preferably, the polymer coating comprises an acrylic polymer chosen from the group consisting of acrylic polymers, methacrylic polymers, ammoniomethacrylate copolymers (especially the Eudragit® RL and RS products), polyacrylates (especially Eudragit® NE) and polymethacrylates (especially Eudragit® E), and mixtures thereof.

The coated amisulpride particle present in the composition according to the invention may also advantageously be obtained via a process comprising at least one step of microencapsulation of the amisulpride particle. The microencapsulation may be obtained via processes that are well known to those skilled in the art, especially via coacervation (phase separation).

Needless to say, the composition according to the invention may comprise a mixture of different coated amisulpride particles chosen from those that may be obtained by microencapsulation, by lipid coating and/or by polymer coating, as described above.

The coated amisulpride particle present in the solid composition according to the invention is preferably between about 21 μm and about 1000 μm in size, in particular between about 31 μm and about 800 μm in size and more particularly between about 91 μm and about 550 μm in size.

The proportion of coating in a coated amisulpride particle is preferably between about 1% and about 50% by weight and more particularly between about 7% and about 35% by weight relative to the total weight of the coated amisulpride particle.

The proportion of amisulpride in a coated amisulpride particle is preferably between about 40% and about 99% by weight relative to the total weight of the coated amisulpride particle.

These respective proportions of coating and of amisulpride may in particular each be determined by a person skilled in the art taking into account the conditions of the process for preparing the coated amisulpride particle, as illustrated in the example below.

The coated amisulpride particle present in the solid composition according to the invention consists of an amisulpride particle preferably between about 20 μm and about 700 μm in size, in particular between about 30 μm and about 600 μm in size and more particularly between about 90 μm and about 500 μm in size, as measured by screening after the process for preparing the amisulpride particle.

The amisulpride particle used for the composition according to the invention, as already defined above, may be prepared according to methods known to those skilled in the art, in particular by granulation or by crystallization.

The granulation may thus be in particular a wet granulation, a granulation with a meltable binder, a granulation by mounting with powders and/or liquids, a rotogranulation or a granulation by extrusion-spheronization. These main routes of granulation are based on the following principles: aggregation or mounting by spraying of powder or liquid.

For the preparation of the amisulpride particles of the solid composition according to the invention, a wet granulation process or an extrusion-spheronization granulation is preferably used.

According to one preferred embodiment of the present invention, the amisulpride particle is a spheroid comprising amisulpride, obtained by granulation.

The amisulpride spheroid thus preferably also comprises at least one spheronization excipient chosen from the group consisting of binders, diluents and surfactants, and mixtures thereof.

Among the binders that are preferred for the granulation of amisulpride, mention may be made of povidone (or polyvinylpyrrolidone; PVP), in particular for wet granulation, such as povidone K30, cellulose-based polymers, in particular for granulation by extrusion-spheronization, such as the hydroxypropylcellulose sold under the name Klucel JF® by the company Aqualon, gums, acrylic polymers, polyethylene glycols (PEG), gelatin and lipid materials. Among the preferred diluents, in particular for granulation by extrusion-spheronization, mention may be made of microcrystalline cellulose such as the product sold under the name Avicel PH101® by the company FMC BioPolymer. Among the surfactants known to those skilled in the art for their use in granulation, in particular by extrusion-spheronization, mention may be made of sodium lauryl sulfate.

According to another embodiment of the present invention, the amisulpride particle may be obtained via a process comprising at least one step of crystallization of amisulpride, such as spherical crystallization.

Taking into account the foregoing text, a person skilled in the art understands that the coating of the coated amisulpride particle as described above advantageously makes it possible to mask the taste of amisulpride, in particular its bitterness. In other words, according to the invention, said at least one coated amisulpride particle consists of an amisulpride particle having at its surface a coating to mask the taste of amisulpride.

Preferably, such a coating does not have a significant effect on the immediate release of amisulpride, i.e. the in vivo release kinetics of the active principle (amisulpride) are not substantially modified with the composition according to the invention compared with that of pharmaceutical compositions with immediate release of amisulpride already used by patients. In other words, according to the invention, preferably, said at least one coated amisulpride particle consists of an amisulpride particle having at its surface a coating to mask the taste of amisulpride without substantially modifying the release of the amisulpride.

Thus, preferably, the composition according to the invention is a composition with immediate release of amisulpride.

According to the invention, the term “composition with immediate release of amisulpride” means a composition that allows an in vitro dissolution of at least 80% by weight of amisulpride in less than 30 minutes in an aqueous 0.1 M hydrochloric acid buffer at 37° C., with a paddle dissolution device (stirring at 100 rpm), in accordance with the in vitro dissolution test described in the European Pharmacopea Section 4.4.

The pharmaceutically acceptable excipient suitable for orodispersible administration of the solid composition according to the invention (also referred to hereinbelow as the “excipient for orodispersible administration”) is preferably chosen from the group consisting of disintegrants, salivation agents and diluents with binding properties, and mixtures thereof.

Among the disintegrants that may be mentioned in particular are crospovidone (or crosslinked polyvinylpyrrolidone), croscarmellose (or crosslinked sodium carboxymethylcellulose), starches and derivatives thereof such as pregelatinized modified starches and carboxymethyl starch, sparingly substituted hydroxypropylcellulose derivatives, and aliginic acid and its salts, and mixtures thereof.

Among the salivation agents that may be mentioned in particular are citric acid and citric acid salts, such as granulated anhydrous citric acid.

Among the diluents with binding properties that may be mentioned in particular are polyols, more particularly mannitol, sorbitol, lactitol, xylitol, erythritol and derivatives, maltitol, and mixtures thereof, especially coatomized xylitol/sorbitol.

The pharmaceutically acceptable excipient suitable for orodispersible administration of the composition is preferably present in a proportion of between about 2% and about 90% by weight and more particularly a proportion of between about 3% and about 85% by weight relative to the total weight of the composition.

More particularly, the respective proportions of disintegrant, of salivation agent and of diluent with binding properties that may constitute the pharmaceutically acceptable excipient suitable for orodispersible administration of the composition according to the invention are preferably:
from about 2% to about 50% by weight (preferably from about 2% to about 20% by weight) of disintegrant;
from about 1% to about 15% by weight (preferably from about 1% to about 5% by weight) of salivation agent; and/or
from about 1% to about 90% by weight of diluent with binding properties, relative to the total weight of the solid composition according to the invention.

Advantageously, the solid composition according to the invention may also comprise, with said at least one coated amisulpride particle and said at least one pharmaceutically acceptable excipient suitable for orodispersible administration of the composition, at least one pharmaceutically acceptable excipient suitable for taste masking (also referred to hereinbelow as “excipient for taste masking”), such as the excipients known to those skilled in the art.

Preferably, this pharmaceutically acceptable excipient suitable for taste masking is chosen from the group consisting of natural flavorings, synthetic flavorings and synthetic sweeteners, and mixtures thereof. Advantageously, a synthetic sweetener in powder or granulated form may be used, especially aspartame.

The proportion of pharmaceutically acceptable excipient suitable for taste masking is preferably between about 0.1% and about 6% by weight and more particularly between about 1% and about 5% by weight relative to the total weight of the composition.

Finally, the solid composition according to the invention may also advantageously comprise at least one pharmaceutically acceptable excipient such as the excipients known to those skilled in the art for the preparation by compression, the conservation and/or the administration of solid pharmaceutical forms (referred to hereinbelow as “excipient for a solid form”), such as tablets or pastilles.

This excipient for a solid form is chosen in particular from the group consisting of diluents, lubricants, flow agents, wetting agents, dyes, pH regulators and binders, and mixtures thereof.

More particularly, among the preferred diluents, mention may be made of microcrystalline cellulose, such as the product sold under the name Avicel PH105®, lactose, such as the product sold under the name Ludipress® and/or glycine such as Glycine BN 500®. Ludipress(200 is an excipient for direct compression, a mixture of lactose (93%), PVP (3.5%) and crosslinked PVP (3.5%). Among the preferred lubricants that may be mentioned is magnesium stearate. Finally, among the preferred flow agents, mention may be made of colloidal silica, such as the product sold under the name Aerosil® by the company Degussa. Among the preferred binders that may be mentioned are polyethylene glycol, povidone (or polyvinylpyrrolidone; PVP), cellulose-based polymers such as hydroxypropylmethylcellulose (HPMC) or hydroxypropylcellulose (HPC), ethylcellulose, acrylic polymers such as those sold under the name Eudragit®, plant oils, gums and gelatin.

As already defined above, the amisulpride included in the pharmaceutical composition according to the invention is chosen from the group consisting of (R,S)-amisulpride, the levorotatory and dextrorotatory isomers of amisulpride, amisulpride derivatives or derivatives of one of its levorotatory and dextrorotatory isomers, these derivatives being chosen from the addition salts with a pharmaceutically acceptable acid, the quaternary ammonium salts and oxides of amisulpride or of one of its levorotatory and dextrorotatory isomers, and mixtures thereof.

Preferably, the amisulpride included in the pharmaceutical composition according to the invention is (R,S)-amisulpride.

Amisulpride is present in the composition according to the invention in a proportion of between about 5% and about 80% by weight relative to the total weight of the composition.

Preferably, the solid composition according to the invention comprises, relative to the total weight of the composition:
about 10% to about 40% by weight of (R,S)-amisulpride in the form of at least one coated amisulpride particle;
about 3% to about 85% by weight of excipient for orodispersible administration;
about 1% to about 5% by weight of excipient for taste masking;
about 1% to about 86% by weight of excipient for a solid form.

The solid composition according to the invention is preferably in the form of a tablet.

Thus, according to one preferred embodiment of the present invention, the solid pharmaceutical composition according to the invention for oral administration of the active principle amisulpride is characterized in that it comprises at least one coated amisulpride particle, at least one pharmaceutically acceptable excipient suitable for orodispersible administration of the composition, and at least one excipient for a solid form, as defined above, and in that it is in the form of a tablet.

The solid composition according to the invention is preferably in the form of an orodispersible tablet comprising amisulpride in a dosage of between 50 and 800 mg and in particular between 100 and 400 mg. More particularly, this orodispersible tablet may comprise amisulpride in a dosage of 100 mg, 200 mg or 400 mg.

Such orodispersible amisulpride tablets may be prepared by direct compression, for example on a SVIAC PR12 rotary machine, preferably with a compression force of less than 600 daN for a 16 mm punch.

The present composition thus more particularly has the advantage of a conserved appearance of a standard solid form, especially that of a tablet, which may, if certain patients desire, be taken with a small amount of water.

The examples that follow are intended to illustrate the present invention and cannot therefore in any way be interpreted as possibly limiting its scope.

### EXAMPLE 1

### Amisulpride Spheroids Obtained by Wet Granulation

Amisulpride particles, in the form of amisulpride spheroids, are prepared by wet granulation using the following components (table 1):

TABLE 1

| Components | Weight percentage |
|---|---|
| Amisulpride¹ | 90 |
| Povidone K30² | 10 |
| Isopropanol | qs granulation |

¹(R,S)-amisulpride manufactured by the company Finorga
²Kollidon ® sold by BASF

The process is performed in the following manner, in a Zanchetta Roto P50 granulator-dryer mixer (MGS). The amisulpride is first mixed with the povidone K30 (binder). The mixture obtained is then subjected to the following successive steps: wetting with isopropanol, granulation, spheronization and drying to remove the solvent.

The amisulpride spheroids thus obtained are calibrated in a screen, to a size of between 125 μm and 500 μm.

A yield of greater than 75% is found for these amisulpride spheroids of between 125 μm and 500 μm in size (the yield is calculated by determining the ratio of the mass of the calibrated spheroids to the sum of the starting masses of amisulpride and of the excipients).

### EXAMPLE 2

### Amisulpride Spheroids Obtained by Extrusion-Spheronization

Amisulpride spheroids, in the form of amisulpride spheroids, are prepared by extrusion-spheronization using the following components (table 2):

TABLE 2

| Components | Weight percentage |
|---|---|
| Amisulpride¹ | 70 |
| Hydroxypropylcellulose² | 1 |
| Cellulose microcrystalline³ | 28.5 |
| Sodium lauryl sulfate | 0.5 |
| Purified water | qs granulation |

¹(R,S)-amisulpride manufactured by Finorga
²Klucel JF ® sold by Hercules
³Avicel PH101 sold by FMC Biopolymer

The process is performed in the following manner. The amisulpride is mixed with the microcrystalline cellulose, the purified water, the hydroxypropylcellulose and the sodium lauryl sulfate in a Diosna granulating mixer. The mixture obtained is then subjected to granulation according to the following steps: extrusion through a Fuji Paudal TDG110 extruder, spheronization in a Fuji Paudal Q400 spheronizer and oven-drying to remove the water. The amisulpride spheroids thus obtained are calibrated to between 125 and 500 μm on a screen.

A yield of greater than 95% is found for the amisulpride spheroids of between 125 μm and 500 μm in size.

### EXAMPLE 3

### Amisulpride Spheroids Coated with Hydrogenated Castor Oil

Coated amisulpride particles, in the form of spheroids coated with hydrogenated castor oil (Cutina HR® sold by Sidobre Sinnova), are prepared using 1500 g of amisulpride spheroids prepared according to example 1. The theoretical coating represents 16.7% by weight relative to the weight of the coated spheroids.

The operating conditions are as follows, in a Glatt fluidized air bed machine of GPCG1 type, with top spray configuration:
total time: 40 min;
air flow rate: 105 Nm³/h;
air atomization temperature: 124° C.;
spraying pressure: 2 bar.

The operating yield is greater than 95%. It is observed on photographs of slices of amisulpride spheroids before and after coating that the coating is uniform and very fine (about 20 μm thick).

A taste test makes it possible to observe that this coating masks the bitter taste of amisulpride.

### EXAMPLE 4

### Amisulpride Spheroids Coated with Gelucire®

Coated amisulpride particles, in the form of spheroids coated with a mixture of monoester and diesters of polyethylene glycol with mono-, di- and triglycerides (Gelucire 50/02® sold by Gattefosseé), are prepared using 1000 g of amisulpride spheroids prepared according to example 1. The theoretical coating represents 16.67% by weight, relative to the weight of the coated spheroids.

The operating conditions are as follows, in a Glatt fluidized air bed machine of GPCG1 type, with top spray configuration:
time: 40 min;
air flow rate: 105 Nm³/h;
atomization air temperature: 65° C.
spraying pressure: 2 bar

The operating yield is greater than 95%.

A taste test makes it possible to observe that this coating masks the bitterness of amisulpride.

### EXAMPLE 5

### Amisulpride Spheroids Coated with Ethylcellulose

Coated amisulpride particles, in the form of amisulpride spheroids coated with a solution comprising 23.40% solids, i.e. 16.54% of ethylcellulose, are prepared starting with 700 g of arisulpride spheroids prepared according to example 1. The ethylcellulose is used in the form of an aqueous dispersion containing
hydroxypropylcellulose (HPMC),
Aquacoat® (sold by FMC BioPolymer), and
Syloid®.

This dispersion is sprayed onto the amisulpride spheroids, in a Glatt fluidized air bed machine of GPCG1 type, of Wuirster configuration.

The coating film has the following composition (table 3):

TABLE 3

| Components | Weight % of solids |
|---|---|
| Aquacoat ® | 78.52 |
| including ethylcellulose | 70.67 |
| HPMC | 15.73 |
| Syloid ® | 5.75 |
| Total: | 100% |

A taste test makes it possible to observe that the bitterness of amisulpride is masked with 22.14% by weight of this coating (including 15.64% of ethylcellulose), relative to the weight of the coated amisulpride spheroids.

### EXAMPLE 6

### Amisulpride Spheroids Coated with an Acrylic Polymer

Coated amisulpride particles, in the form of amisulpride spheroids coated with a solution containing 23% solids, i.e. 15.75% of Eudragit E sold by Röhm & Haas, are prepared using 1000 g of amisulpride spheroids prepared according to example 1.

The polymer Eudragit E® is used in the form of an aqueous dispersion containing magnesium stearate, sodium lauryl sulfate and dibutyl sebacate.

The dispersion is sprayed onto the amisulpride spheroids in a Glatt fluidized air bed machine of GPCGI type, of Wuirster configuration.

The coating film has the following composition (table 4):

TABLE 4

| Components | Weight % of coating solids |
|---|---|
| Eudragit E ® | 68.5 |
| Sodium lauryl sulfate | 4.5 |
| Magnesium stearate | 17 |
| Dibutyl sebacate | 10 |
| Total: | 100% |

A taste test makes it possible to observe that the bitterness of amisulpride is masked with 14.5% by weight of this coating (including 10.25% of Eudragit E® polymer), relative to the weight of the coated amisulpride spheroids.

### EXAMPLE 7

### Orodispersible Amisulpride Tablet

In this example, the coated amisulpride particles are the coated amisulpride spheroids prepared in example 5.

Orodispersible amisulpride tablets are prepared by direct compression, after mixing together the components, on a Sviac PR12 rotary machine with a 16 mm punch and a compression force of less than 600 daN, these tablets thus having the following composition (table 5):

TABLE 5

| Components | Mass (mg) | |
|---|---|---|
| Coated amisulpride spheroids | 460 | |
| (example 5) | | |
| Glycine¹ | 322 | |
| Lactose/PVP/crosslinked PVP² | 276 | |
| Crospovidone³ | 23 | |
| Aspartame | 11.5 | |
| Citric acid | 34.5 | |
| Magnesium stearate | 5.75 | |
| Colloidal silica | 5.75 | |
| Flavorings | 11.5 | |
| Total: | 1150 | mg |

¹Glycine BN 500
²Ludipress ®
³Kollidon CL ®

1. A solid pharmaceutical composition for oral administration of amisulpride, which comprises at least one coated amisulpride particle and at least one pharmaceutically acceptable excipient suitable for an orodispersible administration of the composition.

2. The composition as claimed in claim 1, wherein said at least one coated amisulpride particle consists of an amisulpride particle having at its surface a coating selected from the group consisting of lipid coatings and polymer coatings.

3. The composition as claimed in claim 2, wherein said coating is a lipid coating comprising a lipid material selected from the group consisting of fatty acid mono-, di- and triglycerides, polyglyceryl monoesters and diesters, polyethylene glycol monoesters and diesters, propylene glycol esters, fatty acid esters of sucrose, fatty acids, fatty alcohols, plant waxes, animal waxes, petroleum waxes, synthetic waxes, and mixtures thereof.

4. The composition as claimed in claim 3, wherein said lipid material is selected from the group consisting of hydrogenated castor oil and mixtures of fatty acid mono-, di- and triglycerides with polyethylene glycol monoesters and diesters, and mixtures thereof.

5. The composition as claimed in claim 2, wherein said coating is a polymer coating comprising a polymer selected from the group consisting of cellulose-based polymers, acrylic polymers, vinyl polymers, carboxyvinyl polymers, and mixtures thereof.

6. The composition as claimed in claim 5, wherein said polymer coating comprises a cellulose-based polymer selected from the group consisting of ethylcellulose, hydroxyethylcellulose, hydroxypropylcellulose, hydroxypropylmethylcellulose, carboxymethylcellulose, and mixtures thereof.

7. The composition as claimed in claim 5, wherein said polymer coating comprises an acrylic polymer selected from the group consisting of acrylic polymers, methacrylic polymers, ammoniomethacrylate copolymers, polyacrylates, polymethacrylates, and mixtures thereof.

8. The composition as claimed in claim 1, wherein said at least one coated amisulpride particle is obtained via a process comprising at least one step of microencapsulation of an amisulpride particle.

9. The composition as claimed in claim 1, wherein said at least one coated amisulpride particle is between about 21 μm and about 1000 μm in size.

10. The composition as claimed in claim 1, wherein said at least one coated amisulpride particle consists of an amisulpride particle having a coating at its surface, -the proportion of coating being between about 1% and about 50% by weight relative to the total weight of coated amisulpride particle.

11. The composition as claimed in claim 1, wherein said at least one coated amisulpride particle comprises between about 40% and about 99% by weight of amisulpride relative to the total weight of coated amisulpride particle.

12. The composition as claimed in claim 1, wherein said at least one coated amisulpride particle consists of an amisulpride particle having a coating at its surface, the amisulpride particle being between about 20 μm and about 700 μm in size.

13. The composition as claimed in claim 1, wherein said at least one coated amisulpride particle consists of an amisulpride particle having a coating at its surface, the amisulpride particle being an amisulpride spheroid also comprising at least one spheronization excipient selected from the group consisting of binders, diluents and surfactants, and mixtures thereof.

14. The composition as claimed in claim 13, wherein said at least one spheronization excipient is selected from the group consisting of povidone, hydroxypropylcellulose, microcrystalline cellulose and sodium lauryl sulfate, and mixtures thereof.

15. The composition as claimed in claim 1, wherein said at least one coated amisulpride particle consists of an amisulpride particle having a coating at its surface, the amisulpride particle being obtained via a process comprising at least one step of crystallization of amisulpride.

16. The composition as claimed in claim 1, wherein said at least one pharmaceutically acceptable excipient suitable for orodispersible administration of the composition is selected from the group consisting of disintegrants, salivation agents and diluents with binding properties, and mixtures thereof.

17. The composition as claimed in claim 16, wherein said at least one pharmaceutically acceptable excipient suitable for orodispersible administration of the composition is selected from the group consisting of crospovidone, croscarmellose, starches and derivatives thereof, sparingly substituted hydroxypropylcellulose derivatives, alginic acid and its salts, citric acid and its salts, and polyols, and mixtures thereof.

18. The composition as claimed in claim 1, wherein said at least one pharmaceutically acceptable excipient suitable for orodispersible administration of the composition is present in a proportion of between about 2% and about 90% by weight relative to the total weight of the composition.

19. The composition as claimed in claim 1, which also comprises, in addition to said at least one coated amisulpride particle and said at least one pharmaceutically acceptable excipient suitable for orodispersible administration of the composition, at least one pharmaceutically acceptable excipient suitable for taste masking.

20. The composition as claimed in claim 19, wherein said at least one pharmaceutically acceptable excipient suitable for taste masking is selected from the group consisting of natural flavorings, synthetic flavorings and synthetic sweeteners, and mixtures thereof.

21. The composition as claimed in claim 19, wherein said at least one pharmaceutically acceptable excipient suitable for taste masking is present in a proportion of between about 0.1% and about 6% by weight relative to the total weight of the composition.

22. The composition as claimed in claim 1, which also comprises at least one excipient for a solid form, selected from the group consisting of diluents, lubricants, flow agents, wetting agents, dyes, pH regulators and binders, and mixtures thereof.

23. The composition as claimed in claim 22, wherein said at least one excipient for a solid form is selected from the group consisting of microcrystalline cellulose, lactose, glycine, magnesium stearate, colloidal silica, polyethylene glycol, polyvinylpyrrolidone, cellulose-based polymers, acrylic polymers, plant oils, gums and gelatin, and mixtures thereof.

24. The composition as claimed in claim 1, wherein the amisulpride is selected from the group consisting of (R,S)-amisulpride, the levorotatory and dextrorotatory isomers of amisulpride, amisulpride derivatives and derivatives of one of its levorotatory and dextrorotatory isomers, these derivatives being selected from the addition salts with a pharmaceutically acceptable acid, the quaternary ammonium salts and the oxides, of amisulpride and of its levorotatory and dextrorotatory isomers, and mixtures thereof.

25. The composition as claimed in claim 1, wherein the amisulpride is (R,S)-amisulpride.

26. The composition as claimed in claim 1, wherein the amisulpride is present in a proportion of between about 5% and about 80% by weight relative to the total weight of the composition.

27. The composition as claimed in claim 1, which comprises, relative to the total weight of the composition:
about 10% to about 40% by weight of (R,S)-amisulpride in the form of at least one coated amisulpride particle;
about 3% to about 85% by weight of excipient for orodispersible administration;
about 1% to about 5% by weight of excipient for taste masking;
about 1% to about 86% by weight of excipient for a solid form.

28. The composition as claimed in claim 1, which is in the form of an orodispersible tablet.

29. The composition as claimed in claim 1, which is in the form of an orodispersible tablet comprising amisulpride in a dosage of between 50 and 800 mg.

30. The composition as claimed in claim 1, which affords immediate release of amisulpride.
